(19) United States
(12) Patent Application Publication
(10) Pub. No.: US 2016/0299159 A1
(43) Pub. Date: Oct. 13, 2016

(54) System and Method for Estimating Low-Density Lippoprotein Cholesterol from Standard Lipid Profile

(71) Applicants: Seth S. Martin, Baltimore, MD (US); Steven R. Jones, Baltimore, MD (US)

(72) Inventors: Seth S. Martin, Baltimore, MD (US); Steven R. Jones, Baltimore, MD (US)

(21) Appl. No.: 15/034,692

(22) PCT Filed: Nov. 6, 2014
(86) PCT No.: PCT/US2014/064366

§ 371 Date: May 5, 2016

Related U.S. Application Data
(60) Provisional application No. 61/900,503, filed on Nov. 6, 2013.

(51) Int. Cl. G01N 33/92; G06F 19/00

(52) U.S. Cl. CPC G01N 33/92; G06F 19/704

(57) ABSTRACT

A system and method for estimating a cholesterol associated with low-density lipoprotein are provided. In one aspect, the method includes receiving a lipid profile of a subject, and identifying, from the lipid profile of the subject, a triglyceride concentration and a non-high-density lipoprotein cholesterol concentration. The method also includes selecting, from a database, an adjustable factor based on the triglyceride concentration and the non-high-density lipoprotein cholesterol concentration, wherein the database includes data about a plurality of triglycerides to very low-density lipoprotein cholesterol ratios, and estimating, using a model based on the selected adjustable factor and the lipid profile, a low-density lipoprotein cholesterol. The method further includes generating a report representative of the low-density lipoprotein cholesterol estimate for the subject.

### CROSS-REFERENCE TO RELATED APPLICATIONS

This application is based on, claims priority to, and incorporates herein by reference in its entirety U.S. Provisional Application Ser. No. 61/900,503, filed Nov. 6, 2013, and entitled, “SYSTEMS AND METHODS FOR ESTIMATING LOW-DENSITY LIPPOPROTEIN CHOLESTEROL FROM STANDARD LIPID PROFILE.”

### BACKGROUND OF THE INVENTION

The present disclosure generally relates to systems and methods for estimating low-density lipoprotein cholesterol from lipid profiles derived from a body fluid of a subject, such as blood.

The concentration of cholesterol associated with low-density lipoprotein (LDL-C) is of longstanding clinical and research interest, and the key lipid parameter in national and international clinical practice guidelines. Conventionally, LDL-C may be estimated using the Friedewald equation without need for more sophisticated ultracentrifuge techniques. Specifically, the Friedewald equation was obtained based on an analysis of a small population of 448 patients over 4 decades ago, whereby LDL-C is estimated from the total cholesterol concentration minus high-density lipoprotein cholesterol (HDL-C) concentration minus triglycerides concentration/5. The final term in the equation assumes a fixed ratio of triglycerides to very low-density lipoprotein cholesterol (TG:VLDL-C) of 5:1. As this was approximately the average ratio in the studied population, the equation appeared sufficient at the population-level.

However, applying the population-average factor of 5 to every individual patient is problematic given inter-individual variance in the TG:VLDL-C ratio. Indeed, Friedewald and colleagues noted that simply dividing the triglycerides concentration by 5 does not give a reliable estimate of VLDL-C. Providing further evidence of the variance, the average TG:VLDL-C ratio ranged from 5.2 to 8.9 across clinics in the Lipid Research Clinics Prevalence Study. Although, DeLong et al. proposed modifying the Friedewald formula to a fixed factor of 6, this effectively resets the population average without addressing inter-individual variance in the TG:VLDL-C ratio.

Furthermore, at the time when the Friedewald equation and DeLong et al. modification were proposed, a LDL-C less than 70 mg/dL had not yet been established as an ideal secondary prevention target for treatment of high risk patients. In fact, a LDL-C level in this range was at the low end or outside of the distribution of the original training dataset used in deriving the Friedewald equation. At higher LDL-C concentrations, error in VLDL-C estimation was relatively small with respect to non-HDL-C and actual LDL-C. As such, it was felt that VLDL-C estimation using a simplified computation employing a fixed factor was sufficiently accurate for an era with less developed computing systems.

Considering the above, there continues to be a clear need for accurately estimating LDL-C from the standard lipid profiles without need for more time consuming and expensive technologies that require specialized equipment.

### SUMMARY OF THE INVENTION

The present disclosure overcomes drawbacks of previous technologies by providing a system and method that leverage improvements in computing power and data availability. Specifically, a new approach is introduced to provide a more accurate estimation of low-density lipoprotein cholesterol. This is achieved using a model that includes an adjustable factor, selectable in accordance with data obtained from a standard lipid profile and/or particular subject characteristics, wherein the adjustable factor represents a triglycerides to very low-density lipoprotein cholesterol ratio.

In one aspect of the present disclosure, a method for estimating a cholesterol associated with low-density lipoprotein is provided. The method includes receiving a lipid profile of a subject, and identifying, from the lipid profile of the subject, a triglyceride concentration and a non-high-density lipoprotein cholesterol concentration. The method also includes selecting, from a database, an adjustable factor based on the triglyceride concentration and the non-high-density lipoprotein cholesterol concentration, wherein the database includes data about a plurality of triglycerides to very low-density lipoprotein cholesterol ratios, and estimating, using a model based on the selected adjustable factor and the lipid profile, a low-density lipoprotein cholesterol. The method further includes generating a report representative of the low-density lipoprotein cholesterol estimate for the subject.

In another aspect of the present disclosure, an electronic system for estimating a cholesterol associated with low-density lipoprotein is provided. The system includes an input configured to accept a lipid profile of a subject, a memory having stored therein at least a database having data about a plurality of triglycerides to very low-density lipoprotein cholesterol ratios, and a processor configured to receive a lipid profile of a subject, and identify, from the lipid profile of the subject, a triglyceride concentration and a non-high-density lipoprotein cholesterol concentration. The processor is also configured to select, from the database, an adjustable factor based on the triglyceride concentration and the non-high-density lipoprotein cholesterol concentration, and estimate, using a model based on the selected adjustable factor and the lipid profile, a low-density lipoprotein cholesterol. The system also includes an output configured to provide a report representative of the estimated low-density lipoprotein cholesterol for the subject.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The present invention will hereafter be described with reference to the accompanying drawings, wherein like reference numerals denote like elements.

FIG. 1 is a plan view of an illustrative electronic device configured in accordance with aspects of the present disclosure.

FIG. 2 is a schematic diagram of the illustrative electronic device of FIG. 1 for use in accordance with aspects of the present disclosure.

FIG. 3 is a flowchart setting forth steps of a process for estimating low-density lipoprotein cholesterol for a subject, in accordance with aspects of the present disclosure.

FIG. 4 is a table showing the age, sex, and lipid characteristics of example derivation and validation datasets, in accordance with the present disclosure.

FIG. 5 is a graphical illustration of triglycerides to very low-density lipoprotein cholesterol (TG:VLDL-C) ratios by concentrations of triglycerides and non-high density lipoprotein cholesterol (non-HDL-C), in accordance with the present disclosure.

FIG. 6 is a table showing example median TG:VLDL-C values by non-HDL-C and triglyceride strata, in accordance with the present disclosure.

FIG. 7 is a table showing example concordances in guideline classification by direct LDL-C compared with LDL-C estimates, in accordance with the present disclosure.

FIG. 8 is a graphical illustration of example concordances for LDL-C estimates by triglyceride category, in accordance with the present disclosure.

### DETAILED DESCRIPTION

In clinical and research settings, information related to low-density lipoprotein cholesterol (LDL-C) is used in determining and mitigating risk associated with specific medical conditions, such as cardiac conditions. In particular, there is a strong and graded correlation between LDL-C and the risk of cardiovascular disease. For instance, high levels of LDL-C in the blood is associated with increased risk of atherosclerosis and heart disease. As such, the accuracy of LDL-C measurements can have strong impact on patient outcome. In routine practice, LDL-C is typically estimated using the Friedewald equation, as follows:

[LDL-C]=[TC]−[HDL-C]−[TG]/ƒ (1)

where TC is a total cholesterol concentration, HDL-C is the high-density lipoprotein cholesterol concentration, and TG is the triglycerides concentration obtained from a lipid profile of a subject. In general, the value of ƒ is taken to be 5 in Eqn. 1, under the assumption that the ratio of triglycerides to very low-density lipoprotein cholesterol (TG:VLDL-C) is fixed. However, as will be described, the actual TG:VLDL-C ratio can vary substantially between individuals. Hence, as appreciated from Eqn. 1, inaccuracies in VLDL-C estimation associated with ƒ become increasingly important at lower TC concentrations and higher TG concentrations, since VLDL-C constitutes a greater portion of the equation. As such, errors in VLDL-C estimates introduce larger relative errors in the resulting LDL-C estimate.

Though alternative methods for LDL-C estimation have been previously proposed, none have successfully replaced the Friedewald LDL-C approach in routine practice given their reduced ability in classifying LDL-C based on clinical practice guidelines. Specifically, DeLong et al. proposed an alternative fixed factor, ƒ, namely 6 rather than 5. However, any fixed factor may not account for variance in the TG:VLDL-C ratio. This issue similarly applies to other previous methods, including that of Chen et al. which sets LDL-C equal to 90% of non-HDL-C plus 10% of triglycerides, and de Cordova et al. which takes 75% of non-HDL-C. Moreover, other approaches, such as that of Rao et al. use varied factors that consider only triglyceride, and not cholesterol concentrations, as described by the present invention.

Given these shortcomings, the present disclosure describes an improved approach for accurately estimating LDL-C from a standard lipid profile. Specifically, the system and method described here, rather than assuming a fixed factor for the TG:VLDL-C ratio, instead implement an adjustable factor, selectable from a database based upon particular subject characteristics, such as the triglyceride and non-HDL-C concentrations in the subject's lipid profile. Using a stratified selection criteria, the approach presented herein can obtain results similar to those of Friedewald for patients displaying median TG:VLDL-C values, while generating improved accuracy for patients with high triglycerides and low LDL-C. In addition, methodology described is more concordant with direct LDL-C measurements.

Measurement of LDL-C is of wide interest and deeply ingrained in practice. Guidelines around the globe focus on the LDL-C cut points, including guidelines from the National Heart, Lung, and Blood Institute, Canadian Cardiovascular Society, European Society of Cardiology and European Atherosclerosis Society, the American Heart Association and American College of Cardiology. Some of these guidelines assign the highest level of evidence (class 1A) to LDL-C treatment goals. LDL-C has been a focus in the inclusion criteria of numerous clinical trials, serially quantified during trials, and used as a target for drug titration in some trials. As will become apparent, a strong advantage of the present invention over previous methods occurs particularly in classification of LDL-C concentrations that are less 70 mg/dL, especially in patients with elevated triglycerides.

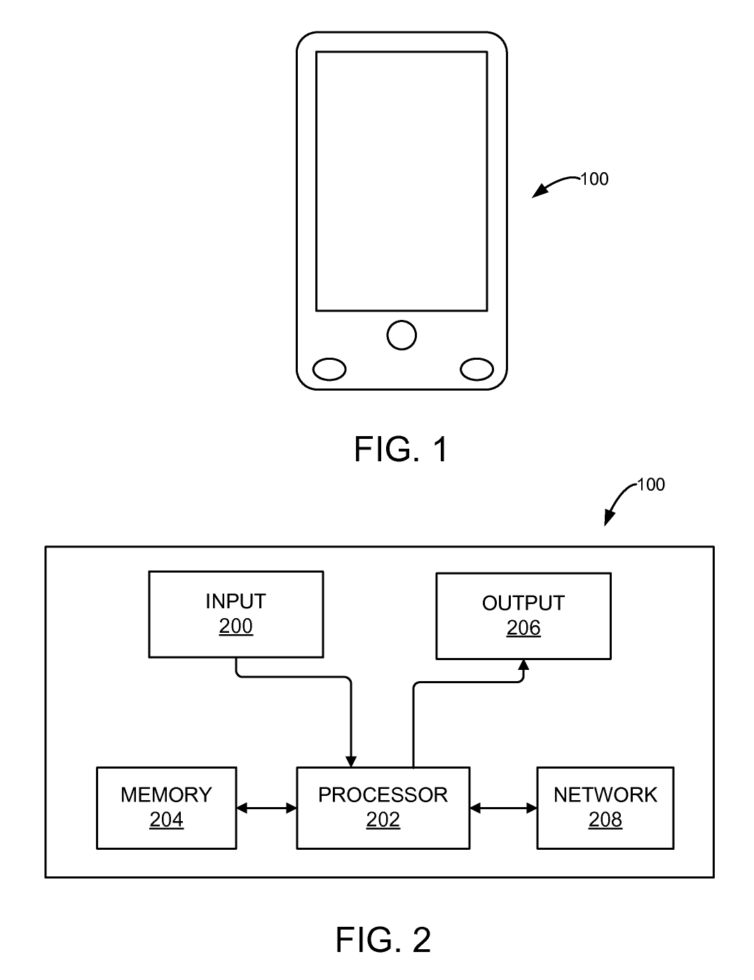

FIG. 1 depicts a plan view of an illustrative electronic system 100 for estimating cholesterol associated with low-density lipoprotein, in accordance with the present disclosure. The system 100 may be a computer, or a part of a computer, system, device, machine, or mainframe, server or may be a mobile, or portable device. For example, the system 100 may be a mobile phone, tablet, or other personal electronic device. In this regard, the system 100 may be a general computing device that may integrate a variety of software and hardware capabilities and functionality.

Referring to FIG. 2, regardless of the particular hardware or software capabilities of the electronic system 100, the system 100 may include some common hardware. For example, the system 100 may include an input 200, which may take any desired or required shape or form, such as communications connections (wired or wireless), buttons, touch displays, and the like. In addition, the input 200 may be provided by way of external readable media. In some aspects, the input 200 may be configured to receive information related particular subject or population characteristics, such as lipid profiles, age, sex, medical condition, or distributions thereof.

The system 100 may include a processor 202 that is designed to carry out any number of actions. As will be described, the processor 202, in addition to other processing tasks, is capable of computing autonomously and semi-autonomously an estimation of low-density lipoprotein cholesterol (LDL-C) from standard lipid profiles using an adjustable factor for the triglycerides to very low-density lipoprotein cholesterol (TG:VLDL-C) ratio. In some aspects, the processor 202 may be configured to receive a lipid profile of a subject and identify, from the lipid profile, a triglyceride concentration, a non-high-density lipoprotein cholesterol concentration, a total cholesterol concentration and so on.

The processor 202 may also be configured to select from a database, using a stratified selection criteria, an adjustable factor related to a triglycerides to very low-density lipoprotein cholesterol ratio. In particular, the processor 202 may select the adjustable factor using subject characteristics, such as triglyceride and the non-high-density lipoprotein cholesterol concentrations obtained from the lipid profile of the subject, as well as the subject's age, a sex, or a medical condition. The processor 202 may then estimate the low-density lipoprotein cholesterol using a model, similar to Eqn. 1, but based on the selected adjustable factor and concentrations obtained from the lipid profile. In some aspects, the processor 202 may also be configured to determine a subject condition, such as a risk for a particular medical condition, based on the estimated low-density lipoprotein cholesterol.

In some aspects, the processor 202 may further be configured to perform a multiple linear regression analysis using data from a population to determine a classification for the above-mentioned database. The classification can include a number of categories associated with different patient variables, such as triglyceride categories, non-high-density lipoprotein cholesterol categories, age categories, sex categories, medical condition categories, and so on. By way of example, the database may include a number of triglyceride categories, for example, 30, where each category corresponds to a range of triglyceride concentration values anywhere between 0 and 15,000 mg/dL, although other values may be possible. Similarly, the database may include a number of non-high-density lipoprotein cholesterol categories, for example, 6, where each category corresponds to a range of non-high-density lipoprotein cholesterol values anywhere between 0 and 300 mg/dL, although other values may be possible. In some aspects, category ranges may be determined based on a distribution of values for each respective variable in the population data.

The system 100 may also include a memory 204 configured to store instructions for operating the processor 202. In some aspects, the memory 204 may also have stored therein a database having, for example, data associated with values triglycerides to very low-density lipoprotein cholesterol ratios, organized into a number of categories, as described above. In addition, the memory 204 may be used by the processor 202 for storing and retrieving data related to a received subject characteristics or lipid profiles. For example, such data may include information related to total cholesterol concentration, high-density lipoprotein cholesterol concentration, triglyceride concentration, as well as a subject's age, a sex, or a medical condition and so on.

In addition, the system 100 may further include an output 206, which may be take any shape or form, as required or desired, including a display configured to provide a clinician or researcher information regarding estimated low-density lipoprotein cholesterol values. In some aspects, the report may include confidence levels related to the estimated low-density lipoprotein cholesterol, as well as indications relation to a subject condition based upon the estimated low-density lipoprotein cholesterol.

In some aspects, the system 100 may further include network communications hardware and software 208, which may be configured to upload and retrieve information or data using wired or wireless, or cloud-based systems. In some aspects, the system 100 may be configured to communicate with an external database, having information related characteristics of a plurality of subjects, as described, via the network 208.

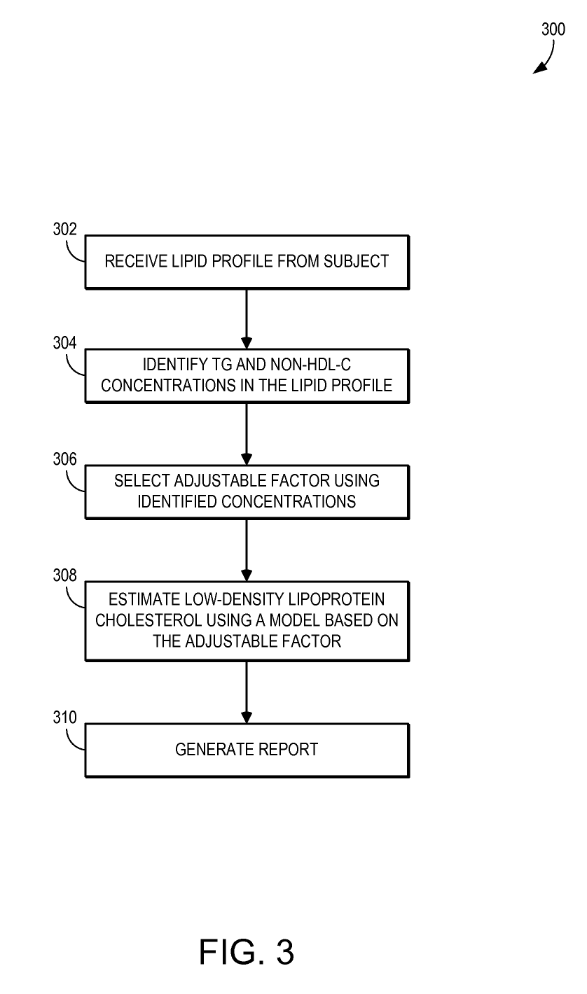

Turning to FIG. 3, a flowchart setting forth steps of a process 300 for estimating low-density lipoprotein cholesterol for a subject, in accordance with aspects of the present disclosure, is shown. The process begins at process block 302, where a lipid profile of a subject is received. In some aspects, subject characteristics, including a subject's age, a sex, or a medical condition, may also be received at process block 302. Then, a triglyceride concentration and a non-high-density lipoprotein cholesterol concentration may be identified from the received lipid profile, as indicated by process block 304. In some aspects, a total cholesterol concentration may also be identified at process block 304 from the lipid profile of the subject.

At process block 306, a stratified selection criteria may be applied to select an appropriate adjustable factor. As described, the adjustable factor may be selected using a database having a number triglycerides to very low-density lipoprotein cholesterol ratios categorized according to a number categories. In particular, the adjustable factor may be selected based on characteristics identified in the lipid profile, such as the triglyceride concentration and the non-high-density lipoprotein cholesterol concentration. In addition, the adjustable factor may also selected based on subject characteristics, including the subject's age, sex, or medical condition.

At process block 308, a low-density lipoprotein cholesterol value may then be estimated, using a model, in accordance with Eqn. 1 above, where the model is based on the selected adjustable factor and parameters obtained from the lipid profile. As such, a report, of any form, representative of the estimated low-density lipoprotein cholesterol for the subject may then be generated at process block 310. In some aspects, the report may include confidence levels related to the estimated low-density lipoprotein cholesterol, as well as indications relation to a subject condition based upon the estimated low-density lipoprotein cholesterol.

The above-described system and method may be further understood by way of example. The example is offered for illustrative purposes only, and is not intended to limit the scope of the present invention in any way. Indeed, various modifications of the invention in addition to those shown and described herein will become apparent to those skilled in the art from the foregoing description and the following example and fall within the scope of the present invention.

### EXAMPLE

### Study Population and Lipid Testing

A consecutive, unselected sample of lipid profiles from the Very Large Database of Lipids (VLDL) was examined Samples were obtained for clinical indications by Atherotech Diagnostics Laboratory (Birmingham, AL) between 2009 and 2011 from 1,350,908 persons living in the United States. To enhance generalizability, children (age<11 years, n=2,129), adolescents (age 11 to <18 years, n=8,165), and adults (age≧18 years, n=1,340,614) were included in the primary analyses. Also both sexes were included, and no patient was excluded based on his or her lipid profile.

Subsequently the contribution of age, sex, and lipid profile characteristics to variance in TG:VLDL-C, as well as the performance of the approach of the present approach for LDL-C estimation within subgroups was evaluated. Lipid distributions in the study sample closely matched those from a population-based survey, the National Health and Nutrition Examination Survey (NHANES) 2007-2008.

Cholesterol concentrations were directly measured by Atherotech's Vertical Auto Profile (VAP) procedure, an inverted rate zonal, single vertical spin, density gradient ultracentrifugation technique that separates lipoprotein sub-fractions, including LDL, VLDL, and HDL. Triglycerides were directly measured using the Abbott ARCHITECT C-8000 system (Abbott Park, Ill.). Analytical performances of direct measures met guideline-established benchmarks. For the VAP procedure, accuracy was monitored by yearly random split-sample comparison with beta quantification at Washington University's Core Laboratory for Clinical Studies (St. Louis, Mo.), and triglyceride measurements were compared with those obtained at the University of Alabama School of Medicine laboratory (Birmingham, Ala.). Between 2009 and 2011, the following correlation coefficients were typically obtained: total cholesterol, 0.99; HDL-C, 0.99; LDL-C, 0.98; VLDL-C, 0.98; triglycerides, 0.99. Between-day intra-assay coefficients of variation were <3.0% for each of these lipid parameters.

### Derivation

Two thirds of patients (n=900,605) were randomly assigned to a derivation dataset. The inter-individual variance was first configured from the distribution of TG:VLDL-C. Subsequently, a multiple linear regression analysis was performed to examine the extent to which TG:VLDL-C was explained by information in the standard lipid profile, age, and sex. Results from this analysis guided the choice of parameters for stratification to determine strata-specific median TG:VLDL-C ratios.

### Validation

One third of patients in the study sample (n=450,303) were randomly assigned to a validation dataset. Patients were grouped by those fulfilling the Friedewald equation criterion of triglycerides <400 mg/dL (n=440,179) and those with triglycerides ≧400 (n=10,124). Friedewald LDL-C (LDL-C_{F}) was estimated as non-HDL-C minus triglycerides/5 in mg/dL. LDL-C estimates using an approach as described by the present invention were derived as non-HDL-C minus triglycerides/adjustable factor in mg/dL, where the adjustable factor was determined as the strata-specific median TG:VLDL-C ratio. Alternative LDL-C estimates were also calculated based on previously proposed formulas. Direct LDL-C (LDL-C_{D}) was subtracted from each LDL-C estimate to determine the absolute difference in their values in mg/dL.

Direct and estimated LDL-C values were classified according to clinical practice guidelines in the United States (<70, 70 to 99, 100 to 129, 130 to 159, 160 to 189, and ≧190 mg/dL) and Europe (<70, 70 to 99, 100 to 154, 155 to 189, and ≧190 mg/dL). Concordance in classification between LDL-C estimates and LDL-C_{D} was examined in the whole study population and subgroups. Odds ratios for discordance in subgroups were calculated using logistic regression. Statistical analyses were performed in Stata v. 11.0 (College Station, Tex.) and logarithmically scaled pseudocolor encoded data density plots were generated in R v. 2.14.1 (Vienna, Austria).

### Results

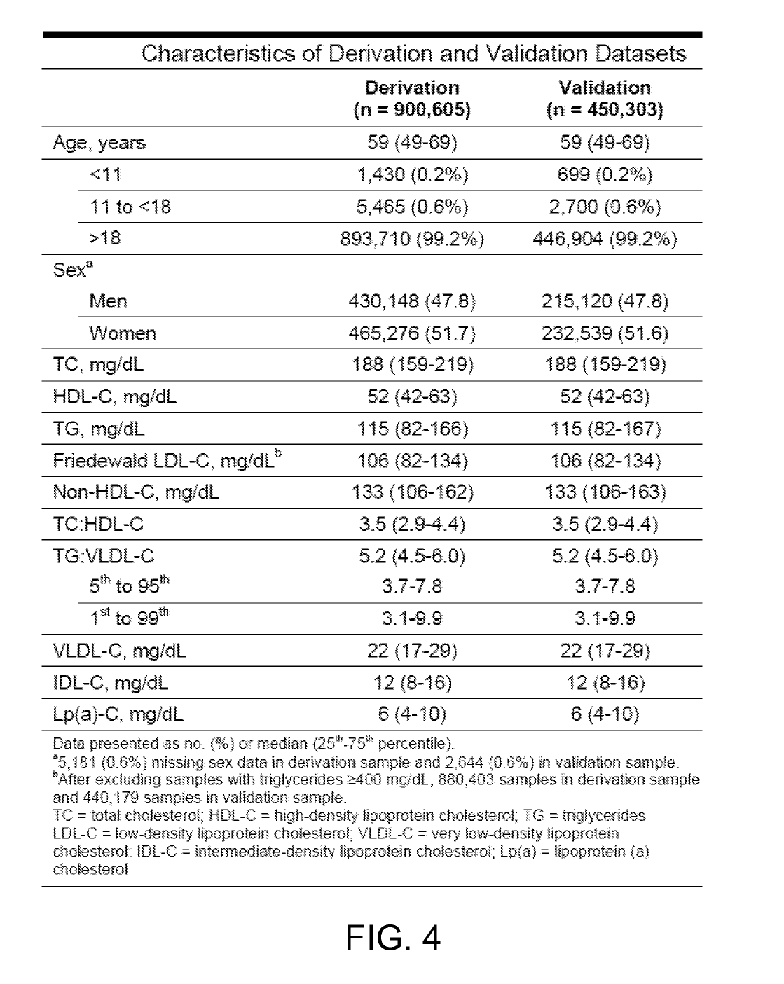

FIG. 4 shows age, sex, and lipid characteristics of the derivation and validation datasets. Patients were generally middle-aged and evenly distributed by sex, with lipid distributions similar to that seen in the general United States population.

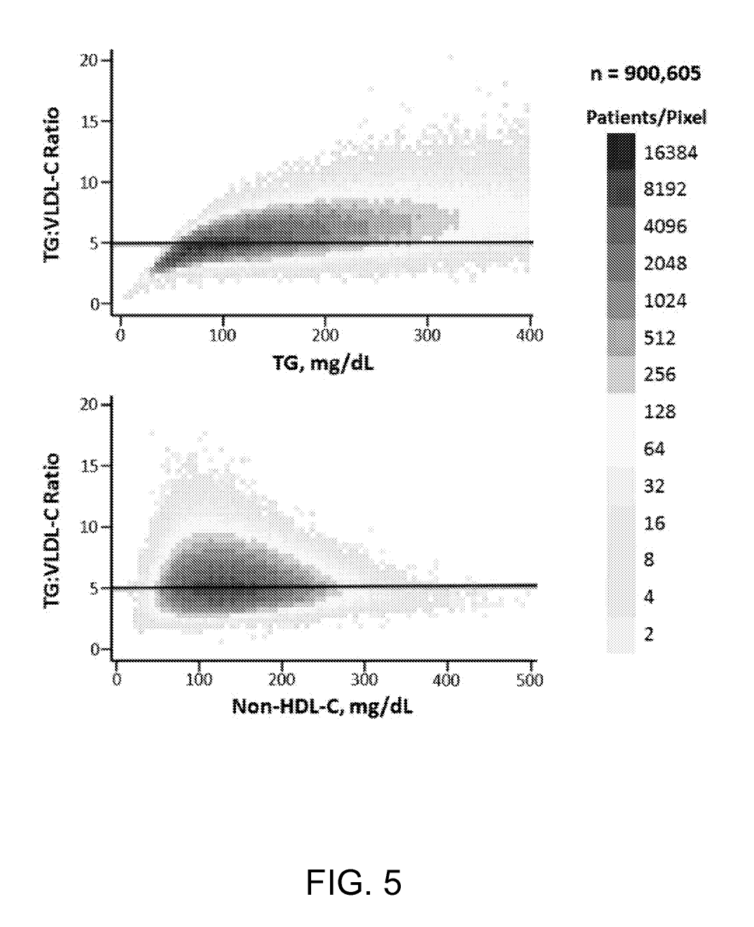

Referring now to FIG. 5, the variance in the TG:VLDL-C ratios by triglyceride and non-HDL-C concentrations is depicted for the derivation dataset (N=900,605). Dark horizontal lines represent a TG:VLDL-C ratio of 5, in accordance with the constant factor used in the Friedewald equation. If the true TG:VLDL-C is greater than 5 (above the line), then the Friedewald formula will tend to underestimated the LDL-C and vice versa. The density of data is expressed by different shades of color, which represents increasing densities of patients per pixel, from light blue to purple.

The median ratio of TG:VLDL-C was 5.2 (25th-75th percentile, 4.5-6.0). Only 1.8% of samples had a TG:VLDL-C exactly equal to 5.0. Approximately one-third had a TG:VLDL-C ratio of 4.5-5.5, and approximately two-thirds had a ratio from 4.0-6.0. The 5th-95th percentile was 3.7-7.8, lst-99th percentile was 3.1-9.9, and full range was 0.4-145. The distribution of the TG:VLDL-C ratio was not normal (skewness 7.1, kurtosis 295.8).

After log-transformation, the TG:VLDL-C ratios were more normally distributed (skewness 0.5, kurtosis 5.6). In regression, the fraction of variance in the log-transformed TG:VLDL-C ratios explained by log-transformed triglycerides was 0.56, and it was 0.65 after adding non-HDL-C to the model and 0.66 if total cholesterol and HDL-C were added as individual components. Further adding age and sex to this model did not materially improve the fraction of variance explained (<0.01 improvement). There was also no material improvement by using ratio variables (total cholesterol to HDL-C, triglycerides to HDL-C, triglycerides to total cholesterol) or using higher degree fractional polynomial regressions rather than linear regression.

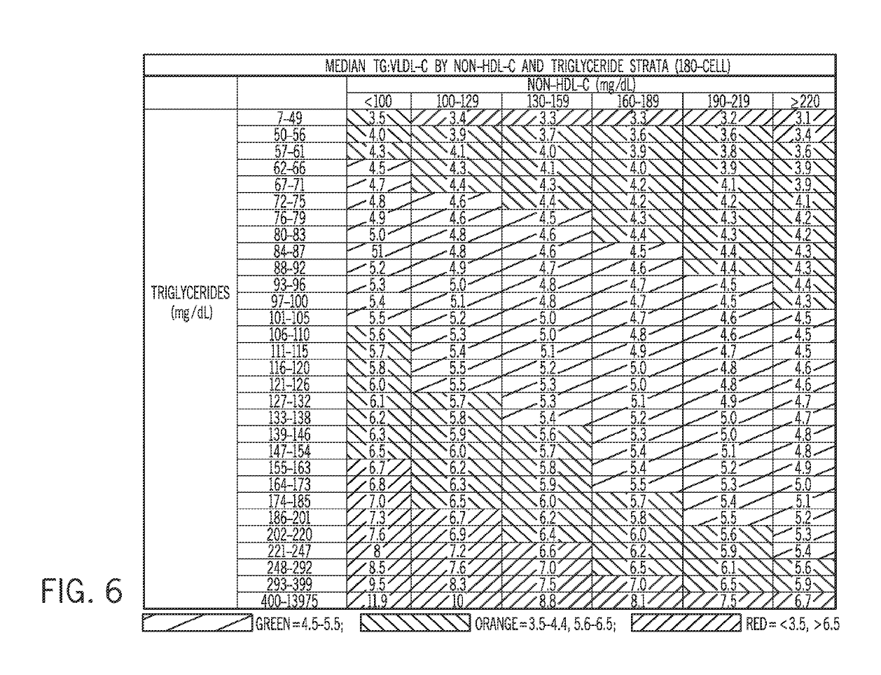

Triglycerides and non-HDL-C explained the variance in TG:VLDL-C about as well as any other permutations of parameters from the standard lipid profile. Therefore, while capturing information on the three core elements from the standard lipid profile, and merging total cholesterol and HDL-C to facilitate a two-dimensional table, focus was maintained on these two parameters for stratification. Varying the number of triglyceride and non-HDL-C strata based on quantiles or accepted cut-points, two-dimensional tables of median TG:VLDL-C ratios were generated using 10, 20, 30, 60, 90, 120, 150, 180, 200, 300, 360, 400, 720, 800, 1000, and 2000 cells. Herein focus was maintained on the 180-cell table of median TG:VLDL-C ratios, which is shown in the table of FIG. 6, although other cell values may be considered.

Strata-specific median TG:VLDL-C ratios from the derivation dataset were applied in the validation dataset to generate novel LDL-C estimates, including those using a 10-cell (LDL-C₁₀), 180-cell (LDL-C₁₈₀), or 360-cell (LDL-C₃₆₀) table. Compared with LDL-C_{F}, these novel LDL-C estimates more closely approximated LDL-C_{D} in patients with triglycerides <400 mg/dL. The median (5th-95th percentile) LDL-C_{F} minus LDL-C_{D} was 0.6 (−15.4−5.0) mg/dL. Compared to LDL-C_{D}, it was 0.0 (−6.0−6.6) for LDL-C₁₀, 0.0 (−5.0−6.4) for LDL-C₁₈0, and 0.0 (−5.0−6.3) for LDL-C₃₆₀. In those with a non-HDL-C <100 mg/dL and triglycerides 100-399 mg/dL, the absolute difference of LDL-C_{F} minus LDL-C_{D} was a median of −4.8 (−18.6−1.0) mg/dL. It was 0.0 (−6.0−6.6) for LDL-C₁₀, 0.0 (−4.7−6.0) for LDL-C₁₈0, and 0.0 (−4.8−6.0) for LDL-C₃₆₀.

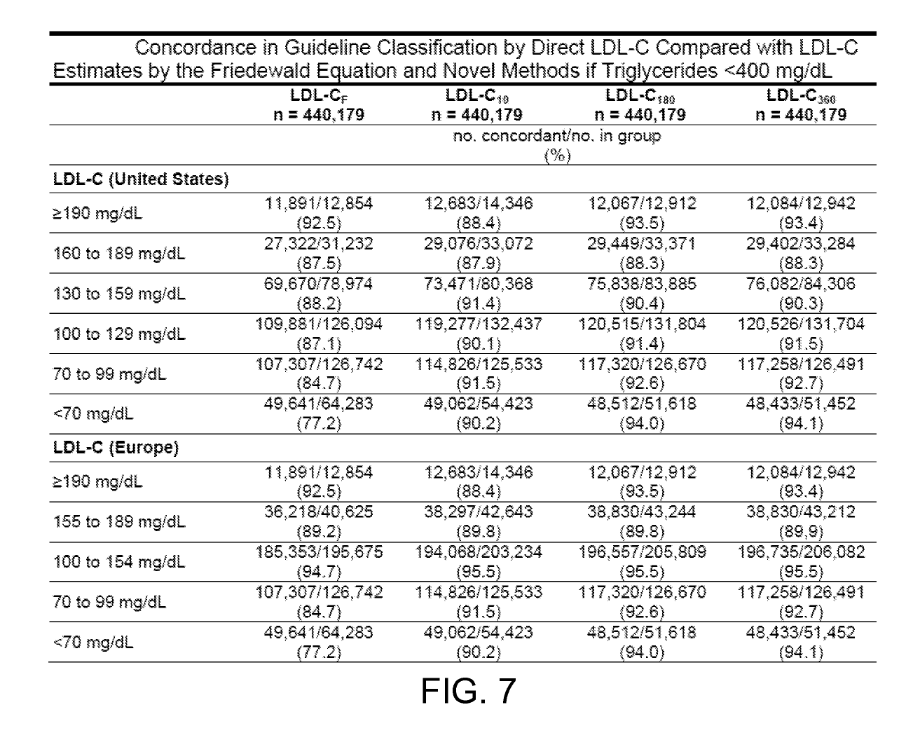

Overall concordance in guideline classification by LDL-C estimates and LDL-C_{D} if triglycerides were <400 mg/dL was 85.3% for LDL-C_{F}, 90.5% for LDL-C₁₀, 91.7% LDL-C₁₈₀, and 91.7% for LDL-C₃₆₀. By individual guideline LDL-C classes, concordances are shown in the table of FIG. 7. The greatest improvement in concordance with novel LDL-C estimates compared with LDL-C_{F} was observed in classifying LDL-C <70 mg/dL. This was particularly for samples with high triglycerides. For example, for patients with an estimated LDL-C <70 mg/dL, direct LDL-C was also <70 mg/dL for novel vs. Friedewald LDL-C in 94.3% vs. 79.9% of samples with triglycerides 100-149 mg/dL, 92.4% vs. 61.3% of samples with triglycerides 150-199 mg/dL, and 84.0% vs. 40.3% of samples with triglycerides of 200-399 mg/dL.

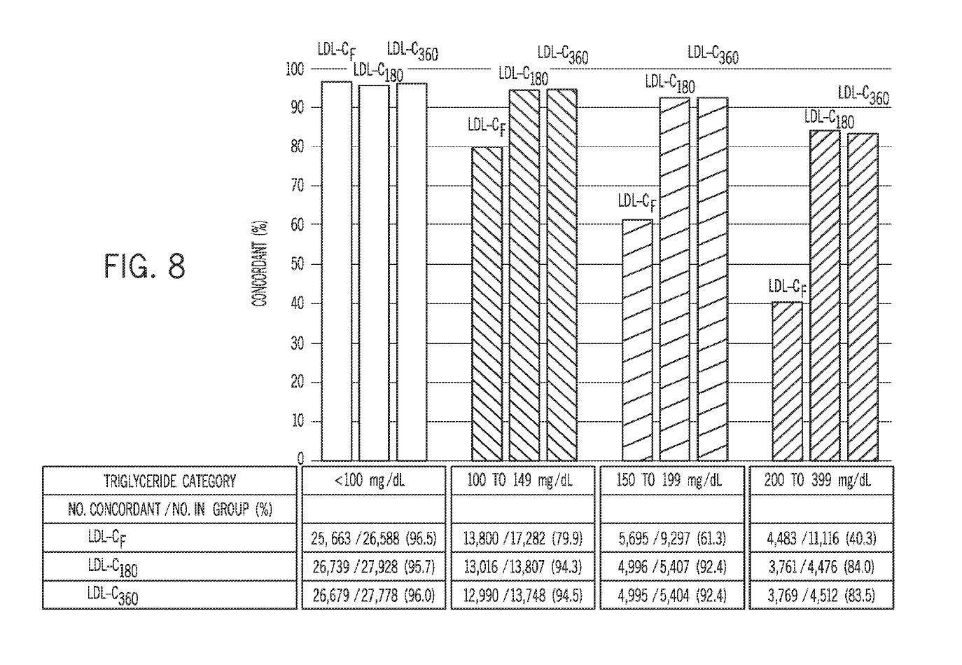

As shown in FIG. 8, LDL-C estimates using LDL-C₃₆₀ performed marginally better than LDL-C₁₈₀ in classifying LDL-C <70 mg/dL when triglycerides were <100 or 100-149 mg/dL, equivalently when triglycerides were 150-199 mg/dL, and marginally worse when triglycerides were >200 mg/dL. Similarly, LDL-C estimates using >360 cells, up to 2000 cells, performed marginally better (<0.1% improvement) in classifying LDL-C <70 mg/dL in some triglyceride categories and marginally worse in others (data not shown).

Adjusting for non-HDL-C and log-transformed triglyceride levels, there was no association of age (adult vs. non-adult, p=0.46) or sex (male vs. female, p=0.91) with discordance in guideline classification between LDL-C₁₈₀ and LDL-C_{D}. In contrast, adjusting for non-HDL-C, those with triglycerides ≧400 mg/dL vs. <400 mg/dL had higher odds of discordance (4.73; 95% CI, 4.53-4.94; p<0.001). Adjusting for non-HDL-C and log-transformed triglyceride, greater discordance was strongly associated with type III Frederickson-Levy dyslipidemia, characterized by excess of remnants and higher cholesterol content of VLDL (odds ratio, 49.9; 95% CI, 38.1-65.3; p<0.001). More modest associations with greater discordance were present for type IIA (odds ratio, 1.05; 95% CI, 1.00-1.10; p=0.049) and type IV dyslipidemia (odds ratio, 1.64; 95% CI, 1.58-1.71; p<0.001), while type IIB dyslipidemia was associated with less discordance (odds ratio, 0.71; 95% CI, 0.66-0.77; p<0.001).

For patients with triglycerides ≧400 mg/dL, concordance with LDL-C_{D} improved using LDL-C₁₈₀ relative to LDL-C_{F} at lower LDL-C levels, although concordance remained modest. Concordance in the setting of Frederickson-Levy dyslipidemias showed results compatible with the above and those reported by Friedewald et al., with the most striking discordance occurring for LDL-C₁₈₀ and LDL-C_{F} in Type III dyslipidemia.

It is noteworthy that patients who undergo a VAP test may be a special population. Despite this, lipid distributions in our sample closely matched a nationally representative population-based survey. It is unknown to what extent patients in our study sample were treated with lipid modifying drug therapies, though Friedewald LDL-C is used ubiquitously in clinical practice for patients regardless of coincident drug therapy. While it is possible that some non-fasting samples may have been included in this study, non-fasting lipid analysis is a common and accepted practice and variance in TG:VLDL-C also exists in completely fasting samples. In addition, our study examines one-time LDL-C measurements. Any measurement has inherent analytical variability. Moreover, while one-time values are commonly used for clinical decision making, guidelines also support serial measurements to calculate a relative change with intervention (e.g., goal of 50% lowering in LDL-C for high risk patients). If LDL-C is over- or under-estimated, then a similar bias may occur in follow-up, rendering relative changes in LDL-C more reliable.

In summary, LDL-C is of wide interest and deeply ingrained in clinical and research practice. Using a very large sample of lipid profiles from a database roughly 3,015 times larger than originally used by Friedewald, a novel approach was derived and validated to estimate LDL-C using an adjustable factor for the triglycerides to very low-density lipoprotein cholesterol (TG:VLDL-C) ratio. This method provides higher fidelity estimates than the tradition Friedewald equation or other prior methods, particularly when classifying LDL-C <70 mg/dL in the presence of high triglycerides.

Guidelines around the globe focus on the LDL-C cut-points of <70 and <100 mg/dL, including guidelines from the National Heart Lung and Blood Institute, Canadian Cardiovascular Society, European Society of Cardiology and European Atherosclerosis Society, and the American Heart Association and American College of Cardiology. Some of these guidelines assign the highest level of evidence (Class IA) to LDL-C treatment goals. LDL-C has been a focus in the inclusion criteria of numerous clinical trials, serially quantified during trials, and used as a target for drug titration in some trials. The Cholesterol Treatment Trialists summarize the totality of evidence for statin therapy as the risk reduction indexed to a 1 mmol/L (39 mg/dL) lowering of LDL-C.

Without resorting to direct assays, estimation using an adjustable factor for estimation of VLDL-C, as described in the present invention, provides a strongly reliable quantification of LDL-C from patient to patient. Of note, however, is that one-third of variance in the TG:VLDL-C ratio may not explained by the standard lipid profile and as such remains a point of caution. This unexplained variance in the TG:VLDL-C ratio represents an intrinsic error in VLDL-C estimation and may be challenging when the clinical question relate to whether a high-risk patient with hypertriglyceridemia has attained a LDL-C <70 mg/dL. Specifically, given the variance in TG:VLDL-C, and hence intrinsic error in VLDL-C estimation, the LDL-C estimates derived from the standard lipid profile may be viewed as complementary to measures that do not depend on VLDL-C estimation, such as non-HDL-C. As such, the methods of the present invention may also benefit from validation using other laboratory techniques.

Although aspects of methods, as described by the present invention, may be limited in some settings of severe hyper-triglyceridemia and type III Frederickson-Levy dyslipidemia, these conditions do not define the full extent of circumstances in which patients may deviate considerably from average. While relatively uncommon in clinical practice, Type III dyslipidemia serum lipid phenotype cannot be reliably identified using the standard lipid panel alone.

Furthermore, another cholesterol-based parameter, non-HDL-C, is not dependent on VLDL-C estimation and has multiple other favorable characteristics. Most notably, non-HDL-C includes cholesterol carried by all atherogenic apolipoprotein B containing lipoproteins, not only that carried by LDL. As such, LDL-C and non-HDL-C may ultimately be best viewed in tandem. At low triglyceride levels, as VLDL-C approaches zero, Friedewald LDL-C approaches non-HDL-C. At triglyceride levels ≧200 mg/dL, guidelines recommend non-HDL-C as a treatment goal. Still, inherent limitations in LDL-C estimation may not be a binary situation confined to patients with triglycerides either ≧200 mg/dL or ≧400 mg/dL, supporting broader consideration of non-HDL-C in practice.

In some contemplated applications, the system and method of the present disclosure may be configured for use in situations where LDL-C is required or desired for clinical or research purposes. Specifically, the system and method described herein may be configured to provide automated or semi-automated estimations of LDL-C using adjustable TG:VLDL-C factors determined using triglyceride and non-HDL-C levels from standard lipid profiles. By way of example, the approach of the present disclosure may be embodied as an personal computer, an online calculator, a mobile or smartphone application, or any other device, system or platform. In other embodiments, the system and method provided may be integrated or utilized in combination with any other devices, systems, or platforms, such as an automated laboratory reporting system and so forth.

The various configurations presented above are merely examples and are in no way meant to limit the scope of this disclosure. Variations of the configurations described herein will be apparent to persons of ordinary skill in the art, such variations being within the intended scope of the present application. In particular, features from one or more of the above-described configurations may be selected to create alternative configurations comprised of a sub-combination of features that may not be explicitly described above. In addition, features from one or more of the above-described configurations may be selected and combined to create alternative configurations comprised of a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

1. A method for estimating a cholesterol associated with low-density lipoprotein, the method comprising:
a) receiving a lipid profile of a subject;
b) identifying, from the lipid profile of the subject, a triglyceride concentration and a non-high-density lipoprotein cholesterol concentration;
c) selecting, from a database, an adjustable factor based on the triglyceride concentration and the non-high-density lipoprotein cholesterol concentration, wherein the database includes data about a plurality of triglycerides to very low-density lipoprotein cholesterol ratios;
d) estimating, using a model based on the selected adjustable factor and the lipid profile, a low-density lipoprotein cholesterol; and
e) generating a report representative of the low-density lipoprotein cholesterol estimate for the subject.

2. The method of claim 1, the method further comprising receiving a subject characteristic that includes at least one of an age, a sex, or a medical condition.

3. The method of claim 2, wherein selecting the adjustable factor at step (c) includes using the subject characteristic.

4. The method of claim 1, the method further comprising performing a multiple linear regression analysis using data from a population to determine a classification for the database.

5. The method of claim 4, wherein the classification includes a plurality of triglyceride categories, wherein each triglyceride category corresponds to a range of the triglyceride concentration values.

6. The method of claim 4, wherein the classification includes a plurality of non-high-density lipoprotein cholesterol categories, wherein each non-high-density lipoprotein cholesterol categories category corresponds to a range of values for the non-high-density lipoprotein cholesterol concentration.

7. The method of claim 1, wherein the method further comprises identifying a total cholesterol concentration from the lipid profile of the subject.

8. The method of claim 7, wherein the model providing the low-density lipoprotein cholesterol estimate computes
[LDL-C]=[TC]−[HDL-C]−[TG]/ƒ
where TC is the total cholesterol concentration, HDL-C is a high-density lipoprotein cholesterol concentration, TG is the triglyceride concentration, and ƒ is the adjustable factor selected at step (c).

9. An electronic system for estimating a cholesterol associated with low-density lipoprotein, the system comprising:
an input configured to accept a lipid profile of a subject;
a memory having stored therein at least a database having data about a plurality of triglycerides to very low-density lipoprotein cholesterol ratios;
a processor configured to:
i) receive a lipid profile of a subject;
ii) identify, from the lipid profile of the subject, a triglyceride concentration and a non-high-density lipoprotein cholesterol concentration;
iii) select, from the database, an adjustable factor based on the triglyceride concentration and the non-high-density lipoprotein cholesterol concentration;
iv) estimate, using a model based on the selected adjustable factor and the lipid profile, a low-density lipoprotein cholesterol; and
an output configured to provide a report representative of the estimated low-density lipoprotein cholesterol for the subject.

10. The system of claim 9, wherein the processor is further configured to receive a subject characteristic that includes at least one of an age, a sex, or a medical condition from the input.

11. The system of claim 10, wherein the processor is further configured to select the adjustable factor at step (iii) using the subject characteristic.

12. The system of claim 9, wherein the processor is further configured to perform a multiple linear regression analysis using data from a population to determine a classification for the database.

13. The system of claim 12, wherein the classification includes a plurality of triglyceride categories, wherein each triglyceride category corresponds to a range of the triglyceride concentration values.

14. The system of claim 12, wherein the classification includes a plurality of non-high-density lipoprotein cholesterol categories, wherein each non-high-density lipoprotein cholesterol category corresponds to a range of values for the non-high-density lipoprotein cholesterol concentration.

15. The system of claim 9, wherein the processor is further configured to identify a total cholesterol concentration from the lipid profile of the subject.

16. The system of claim 15, wherein the model providing the low-density lipoprotein cholesterol estimate at step (iv) computes
[LDL-C]=[TC]−[HDL-C]−[TG]/ƒ
where TC is the total cholesterol concentration, HDL-C is a high-density lipoprotein cholesterol concentration, TG is the triglyceride concentration, and ƒ is the adjustable factor selected at step (iii).
